(19) United States
(12) Patent Application Publication
(10) Pub. No.: US 2004/0116497 A1
(43) Pub. Date: Jun. 17, 2004

(54) Chromane derivatives, process for their preparation and their use as antitumor agents

(76) Inventors: Gabriella Traquandi, Milan (IT); Maria Gabriella Brasca, Cusago (IT); Paolo Orsini, Gallarte (IT); Claudia Piutti, Nerviano (IT); Anna Vulpetti, Brugherio (IT); Paolo Pevarello, Pavia (IT)

Correspondence Address: Dwayne L Mason, Akin Gump Strauss Hauer & Field, Suite 1900, 711 Louisiana, Houston, TX, 77002, US

(21) Appl. No.: 10/466,412

(22) PCT Filed: Jan. 17, 2002
(86) PCT No.: PCT/EP02/00524

(30) Foreign Application Priority Data
Jan. 26, 2001 (US) 09769441

(51) Int. Cl. A61K 31/416

(52) U.S. Cl. 514/406

(57) ABSTRACT

Compounds which are chromane derivatives of formula (I), pharmaceutically acceptable salts, process for their preparation and pharmaceutical compositions thereof are disclosed, as set forth in the specification; these compounds are useful in therapy in the treatment of cell proliferative disorders, e.g. cancer, associated with an altered cell cycle dependent kinase activity.

### BACKGROUND OF THE INVENTION

1. Field of the Invention

The present invention relates to chromane derivatives and, more particularly, to chromane derivatives and analogues thereof, to a process for their preparation, to pharmaceutical compositions comprising them, and to their use as therapeutic agents, particularly in the treatment of cancer and cell proliferative disorders.

2. Discussion of the Background

Several cytotoxic drugs such as, e.g., fluorouracil (5-FU), doxorubicin and camptothecins, damage DNA or affect cellular metabolic pathways and thus cause, in many cases, an indirect block of the cell cycle. Therefore, by producing an irreversible damage to both normal and tumor cells, these agents result in a significant toxicity and side-effects. In this respect, compounds capable of functioning as highly specific antitumor agents by selectively leading to tumor cell arrest and apoptosis, with comparable efficacy but reduced toxicity than the currently available drugs, are desirable.

It is well known that progression through the cell cycle is governed by a series of checkpoint controls, otherwise referred to as restriction points, which are regulated by a family of enzymes known as the cyclin-dependent kinases (cdk). In turn, the cdks themselves are regulated at many levels such as, for instance, binding to cyclins.

The coordinated activation and inactivation of different cyclin/cdk complexes is necessary for normal progression through the cell cycle. Both the critical G1-S and G2-M transitions are controlled by the activation of different cyclin/cdk activities. In G1, both cyclin D/cdk4 and cyclin E/cdk2 are thought to mediate the onset of S-phase. Progression through S-phase requires the activity of cyclin A/cdk2 whereas the activation of cyclin A/cdc2 (cdk1) and cyclin B/cdc2 are required for the onset of metaphases. For a general reference to cyclins and cyclin-dependent kinases see, for instance, Kevin R. Webster et al, in Exp. Opin. Invest. Drugs, 1998, Vol. 7(6), 865-887. Checkpoint controls are defective in tumor cells due, in part, to disregulation of cdk activity. For example, altered expression of cyclin E and cdks has been observed in tumor cells, and deletion of the cdk inhibitor p27 KIP gene in mice has been shown to result in a higher incidence of cancer.

Increasing evidence supports the idea that the cdks are rate-limiting enzymes in cell cycle progression and, as such, represent molecular targets for therapeutic intervention. In particular, the direct inhibition of cdk/cyclin kinase activity should be helpful in restricting the unregulated proliferation of a tumor cell.

### SUMMARY OF THE INVENTION

It is an object of the invention to provide compounds which are useful in treating cell proliferative disorders associated with an altered cell cycle dependent kinase activity. It is another object to provide compounds which have cdk/cyclin kinase inhibitory activity. It is another object of the invention to provide compounds which are useful in therapy as antitumor agents but lack, in terms of both toxicity and side effects, the drawbacks associated with currently available antitumor drugs discussed above.

The present inventors have now discovered that certain chromane derivatives and analogues thereof, also referable to as pyrazolyl-aminocarbonyl-chromane derivatives but hereinafter solely referred to as chromanes, are endowed with cdk/cyclin kinase inhibitory activity and are thus useful in therapy as antitumor agents whereas lacking the above toxicity and side effects.

More specifically, the chromanes of the invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma.

Due to the key role of cdks in regulating cellular proliferation, the chromane derivatives of the invention are also useful in the treatment of a variety of cell proliferative disorders such as, for example, benign prostate hyperplasia, familial adenomatosis polyposis, neurofibromatosis, psoriasis, vascular smooth cell proliferation associated with atherosclerosis, pulmonary fibrosis, arthritis, glomerulonephritis and post-surgical stenosis and restenosis.

The compounds of the invention may be useful in treatment of Alzheimer's disease, as suggested by the fact that cdk5 is involved in the phosphorylation of tau protein (J. Biochem. 117, 741-749, 1995).

The compounds of this invention, as modulators of apoptosis, may also be useful in the treatment of cancer, viral infections, prevention of AIDS development in HIV-infected individuals, autoimmune diseases and neurodegenerative disorders.

The compounds of this invention may be useful in inhibiting tumor angiogenesis and metastasis.

The compounds of the invention may also act as inhibitor of other protein kinases, e.g., protein kinase C, Met, PAK4, PAK-5, ZC-1, STLK-2, DDR-2, Aurora, Aurora 2, Bub-1, PLK, Chk1, Chk2, Her2, raf1, MEK1, MAP kinase, EGF receptor, PDGF receptor, IGF receptor, PI3 kinase, weel kinase, Src, Abl, and thus be effective in the treatment of diseases associated with other protein kinases.

The compounds of the invention are also useful in the treatment and prevention of radiotherapy-induced or chemotherapy-induced alopecia.

Accordingly, the present invention provides a method for treating cell proliferative disorders associated with an altered cell cycle dependent kinase activity, by administering to a mammal in need thereof an effective amount of a chromane derivative represented by formula

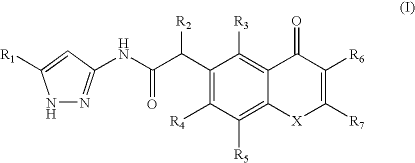

wherein

R₁ is a C₃-C₆ cycloalkyl group optionally substituted by a straight or branched C₁-C₆ alkyl or by aryl C₁-C₆ alkyl group;

R₂ is a hydrogen atom or a straight or branched C₁-C₆ alkyl or C₂-C₄ alkenyl group, each of which being optionally substituted by hydroxy, C₁-C₆ alkoxy, amino or C₁-C₆ alkylamino;

R₃, R₄ and R₅ are, each independently, hydrogen, halogen, hydroxy, amino or straight or branched C₁-C₆ alkyl, C₁-C₆ alkoxy or C₁-C₆ alkylamino;

R₆ and R₇ are, each independently, hydrogen, hydroxy, amino, aminocarbonyl, ureido, guanidyl, pyrrolidinyl optionally substituted by oxo groups, straight or branched C₁-C₆ alkyl optionally substituted by hydroxy or amino groups, straight or branched C₁-C₆ alkoxy, aryl or arylcarbonyl optionally substituted by halogen, hydroxy, amino, straight or branched C₁-C₆ alkyl or C₁-C₆ alkoxy groups, or a group selected from alkylcarbonyl, alkylamino, alkylaminocarbonyl or arylalkyloxy wherein alkyl stands for straight or branched C₁-C₆ alkyl;

X is an oxygen or sulfur atom or represents a group —N(R₈)— wherein R₈ is hydrogen or a straight or branched C₁-C₆ alkyl or C₂-C₄ alkenyl group, each of which being optionally substituted by hydroxy, amino, C₁-C₆ alkoxy or C₁-C₆ alkylamino;

or a pharmaceutically acceptable salt thereof;

provided that the compound of formula (I) is other than N-(5-cyclopropyl-1H-pyrazol-3-yl)-2-[2-(4-methoxyphenyl)-4-oxo-4H-chromen-6-yl]acetamide.

In a preferred embodiment of the method described above, the cell proliferative disorder is selected from the group consisting of cancer, Alzheimer's disease, viral infections, auto-immune diseases and neurodegenerative disorders.

Specific types of cancer that may be treated include carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer, and Kaposi's sarcoma.

In another preferred embodiment of the method described above, the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis, glomerulonephritis and post-surgical stenosis and restenosis.

In addition, the inventive method provides tumor angiogenesis and metastasis inhibition. The inventive method may also provide cell cycle inhibition or cdk/cyclin dependent inhibition.

In addition to the above, the method object of the present invention provides treatment and prevention of radiotherapy-induced or chemotherapy-induced alopecia.

The present invention also provides a chromane derivative represented by formula

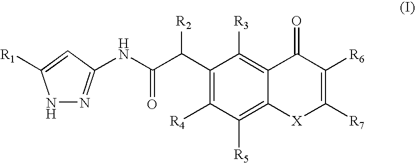

wherein

R₁ is a C₃-C₆ cycloalkyl group optionally substituted by a straight or branched C₁-C₆ alkyl or by aryl C₁-C₆ alkyl group;

R₂ is a hydrogen atom or a straight or branched C₁-C₆ alkyl or C₂-C₄ alkenyl group, each of which being optionally substituted by hydroxy, C₁-C₆ alkoxy, amino or C₁-C₆ alkylamino;

R₃, R₄ and R₅ are, each independently, hydrogen, halogen, hydroxy, amino or straight or branched C₁-C₆ alkyl, C₁-C₆ alkoxy or C₁-C₆ alkylamino;

P₆ and R₇ are, each independently, hydrogen, hydroxy, amino, aminocarbonyl, ureido, guanidyl, pyrrolidinyl optionally substituted by oxo groups, straight or branched C₁-C₆ alkyl optionally substituted by hydroxy or amino groups, straight or branched C₁-C₆ alkoxy, aryl or arylcarbonyl optionally substituted by halogen, hydroxy, amino, straight or branched C₁-C₆ alkyl or C₁-C₆ alkoxy groups, or a group selected from alkylcarbonyl, alkylamino, alkylaminocarbonyl or arylalkyloxy wherein alkyl stands for straight or branched C₁-C₆ alkyl;

X is an oxygen or sulfur atom or represents a group —N(R₈)— wherein R₈ is hydrogen or a straight or branched C₁-C₆ alkyl or C₂-C₄ alkenyl group, each of which being optionally substituted by hydroxy, amino, C₁-C₆ alkoxy or C₁-C₆ alkylamino;

or a pharmaceutically acceptable salt thereof;

provided that the compound of formula (I) is other than N-(5-cyclopropyl-1H-pyrazol-3-yl)-2-[2-(4-methoxyphenyl)4-oxo-4H-chromen-6-yl]acetamide.

The present invention also includes methods of synthesizing the chromane derivatives represented by formula (I).

A pharmaceutical composition comprising the chromane derivatives of formula (I) is also included in the present invention.

A more complete appreciation of the invention and many of the attendant advantages thereof will be readily obtained as the same becomes better understood by reference to the following detailed description.

### DETAILED DESCRIPTION OF THE INVENTION

Several chromane derivatives are known in the art, for instance as synthetic intermediates or even as pharmacologically active agents.

Among them are, as an example, 4-oxo-4H-1-benzopyran-6-carboxylic acids which have been described as antiallergic agents (see, for a reference, Eur. J. Med. Chem.-Chimica Therapeutica, 1978-13, No. 1, 33-39).

Carbonylamino-pyrazole derivatives are also known in the art, for instance as pesticides, herbicides or even as therapeutic agents. Among them are, as an example, heteroaryl-pyrazoles active as p38 kinase inhibitors (WO 98/52941, G.D. Searle and Co.) and 3-amino-pyrazoles active as protein kinase inhibitors (WO 96/14843, COR Therapeutics, Inc.).

A class of carbonylamino-pyrazoles endowed with cyclin dependent kinase inhibitory activity, also comprising N-(5-cyclopropyl-1H-pyrazol-3-yl)-2-[2-(4-methoxyphenyl)-4-oxo-4H-chromen-6-yl]acetamide, are also disclosed in the international patent application WO 01/12189 (PCT/US00/06699, filed on May 5, 2000), in the name of Pharmacia & Upjohn S.p.A and Pharmacia & Upjohn Co., which is herewith incorporated by reference.

As it will be readily appreciated, the unsubstituted ring nitrogen-pyrazoles in the compounds of the invention are known to rapidly equilibrate, in solution, as admixtures of both tautomers:

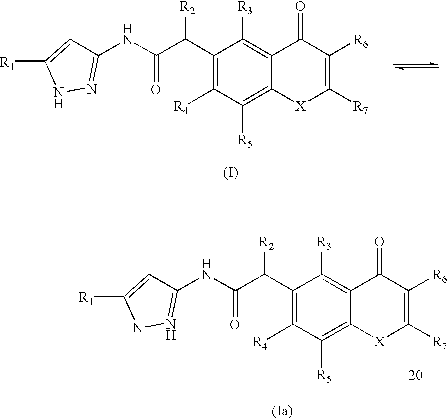

Accordingly, in the present invention and unless specifically noted otherwise, where only one tautomer is indicated for the compounds of formula (I), the other (Ia) is also within the scope of the invention.

The compounds of formula (I) may have asymmetric carbon atoms and may therefore exist either as racemic admixtures or as individual optical isomers which are all within the scope of the present invention.

Likewise, the use as an antitumor agent of all the possible isomers and their admixtures and of both the metabolites and the pharmaceutically acceptable bio-precursors (otherwise referred to as pro-drugs) of the compounds of formula (I) are also within the scope of the present invention.

As used herein, unless otherwise specified, the term C₃-C₆ cycloalkyl includes cyclopropyl, cyclobutyl, cyclopentyl or cyclohexyl.

The terms straight or branched C₁-C₆ alkyl or alkoxy groups include, for instance, methyl, ethyl, n-propyl, isopropyl, n-butyl, isobutyl, tert-butyl, sec-butyl, n-pentyl, n-hexyl, methoxy, ethoxy, n-propoxy, isopropoxy, n-butoxy, isobutoxy, tert-butoxy, sec-butoxy, n-pentyloxy, n-hexyloxy, and the like.

The term straight or branched C₂-C₄ alkenyl includes vinyl, allyl, 1-propenyl, isopropenyl, 1-butenyl, 2-butenyl, 3-butenyl, and the like.

The term aryl includes either carbocyclic or heterocyclic hydrocarbons with 1 or 2 ring moieties either fused or linked to each other by a single bond, wherein at least one of them is a 5 or 6 membered aromatic ring.

Examples of aryl groups are, for instance, phenyl, biphenyl, α- or β-naphthyl, dihydronaphthyl, thienyl, benzothienyl, furyl, benzofuranyl, dihydrobenzofuranyl, chromenyl, pyrrolyl, imidazolyl, pyrazolyl, thiazolyl, isothiazolyl, oxazolyl, isoxazolyl, pyridyl, pyrazinyl, pyrimidinyl, pyridazinyl, indolyl, isoindolyl, purinyl, quinolyl, isoquinolyl, dihydroquinolyl, quinoxalinyl, benzodioxolyl, indanyl, indenyl, triazolyl, tetrazolyl and the like.

The term heterocycle, hence encompassing heteroaromatic rings also referred to as aryl group, includes a 5 to 6 membered saturated or unsaturated carbocycle wherein one or more carbon atoms are replaced by one or more atoms selected from nitrogen, oxygen and sulfur.

Examples of saturated or partly unsaturated heterocycles are, for instance, pyran, pyrrolidine, pyrrolne, imidazoline, imidazolidine, dihydrofuran, tetrahydrofuran, 1,3-dioxolane, piperidine, piperazine, morpholine and the like.

Unless otherwise specified, the term pyrrolidinyl is herewith intended to comprise pyrrolidinyl groups such as 1-, 2- or 3-pyrrolinyl, which are optionally further substituted by oxo groups such as, for instance, 2-oxo-pirrolidin-5-yl.

Unless otherwise indicated, the term halogen atom includes fluorine, chlorine, bromine and iodine.

From the foregoing, any of the terms such as alkylamino, alkylaminocarbonyl, alkylcarbonyl, arylcarbonyl, arylalkyl, arylalkyloxy and the like, include groups wherein the alkyl and aryl moieties are as described above.

Pharmaceutically acceptable salts of the compounds of formula (I) include the acid addition salts with inorganic or organic acids, e.g. nitric, hydrochloric, hydrobromic, sufliric, perchloric, phosphoric, acetic, trifluoroacetic, propionic, glycolic, lactic, oxalic, malonic, malic, maleic, tartaric, citric, benzoic, cinnamic, mandelic, methansulphonic, isethionic and salicylic acid, as well as the salts with inorganic or organic bases, e.g., alkali or alkaline-earth metals, especially sodium, potassium, calcium or magnesium hydroxides, carbonates or bicarbonates, acyclic or cyclic amines, preferably methylamine, ethylamine, diethylamine, triethylamine or piperidine.

Preferred compounds of the invention are the compounds of formula (I) wherein R₁ is a C₃-C₆ cycloalkyl group; R₂ is hydrogen or a straight or branched C₁-C₄ alkyl group; R₃, R₄ and R₅ are, each independently, hydrogen, halogen or a straight or branched C₁-C₆ alkyl or C₁-C₆ alkoxy group; R₆ and R₇ are, each independently, hydrogen, hydroxy, amino, aminocarbonyl, ureido, guanidyl, straight or branched C₁-C₄ alkyl optionally substituted by hydroxy or amino, straight or branched C₁-C₄ alkoxy, straight or branched C₁-C₄ alkylcarbonyl or alkylaminocarbonyl, arylcarbonyl, aryl C₁-C₄ alkyloxy, or aryl optionally substituted by halogen, amino, hydroxy or straight or branched C₁-C₄ alkyl or C₁-C₄ alkoxy, X is oxygen, sulfur or a group —N(R₈)— wherein R₈ is as above defined.

More preferred compounds, within this class, are those of formula (I) wherein R₁ is cyclopropyl; R₂ is hydrogen or methyl; R₃, R₄ and R₅ are, each independently, hydrogen, halogen, methyl or methoxy; R₆ and R₇ are, each independently, hydrogen or aryl groups optionally substituted by halogen, amino, hydroxy or straight or branched C₁-C₄ alkyl or C₁-C₄ alkoxy groups; X is oxygen, sulfur or a group —N(R₈)— wherein R₈ is as above defined.

Even more preferred compounds of the invention are the derivatives of formula (I) wherein R₁ is cyclopropyl; R₂ is hydrogen or methyl; R₃, R₄ and R₅ are, each independently, hydrogen, fluorine, chlorine or bromine or a methoxy group; R₆ and R₇ are, each independently, hydrogen or aryl optionally further substituted as above indicated, wherein the aryl is selected from the group consisting of phenyl, pyrrolyl, pyrazolyl, imidazolyl, furyl, thienyl or pyridyl; X is oxygen, sulfur or a group —N(R₈)— wherein R₈ is as above defined.

According to a preferred embodiment of the invention, within the compounds of formula (I) above defined, X is an oxygen atom.

According to another preferred embodiment of the invention, within the compounds of formula (I), X is a group —N(R₈)— wherein R₈ is hydrogen or a straight or branched C₁-C₆ alkyl or C₂-C₄ alkenyl group, each of which being optionally substituted by hydroxy, amino, C₁-C₆ alkoxy or C₁-C₆ alkylamino.

Even more preferably, within the compounds of formula (I) of the invention X is a group —N(R₈)— wherein R₈ is a hydrogen atom.

Examples of preferred compounds of the invention, which may be in the form of pharmaceutically acceptable salts, for instance as hydrochloride or hydrobromide salts, include the following:

1. N-(5-cyclobutyl-1H-pyrazol-3-yl)-2-[2-(4-methoxyphenyl)-4-oxo-4H-chromen-6-yl]acetamide;

2. N-(5-cyclopentyl-1H-pyrazol-3-yl)-2-[2-(4-methoxyphenyl)-4-oxo-4H-chromen-6-yl]acetamide;

3. N-(5-cyclopropyl-1H-pyrazol-3-yl)-2-[2-(4-methoxyphenyl)-4-oxo-1,4-dihydro-6-quinolinyl]acetamide;

4. N-(5-cyclobutyl-1H-pyrazol-3-yl)-2-[2-(4-methoxyphenyl)-4-oxo-1,4 dihydro-6-quinolinyl]acetamide;

5. N-(5-cyclopentyl-1H-pyrazol-3-yl)-2-[2-(4-methoxyphenyl)-4-oxo-1,4-dihydro-6-quinolinyl]acetamide;

6. N-(5-cyclopropyl-1H-pyrazol-3-yl)-2-[2-(4-methoxyphenyl)-4-oxo-4H-chromen-6-yl]propanamide;

7. N-(5-cyclobutyl-1H-pyrazol-3-yl)-2-[2-(4-methoxyphenyl)-4-oxo-4H-chromen-6-yl]propanamide;

8. N-(5-cyclopentyl-1H-pyrazol-3-yl)-2-[2-(4-methoxyphenyl)4-oxo-4H-chromen-6-yl]propanamide;

9. N-(5-cyclopropyl-1H-pyrazol-3-yl)2-[2-(4-methoxyphenyl)-4-oxo-1,4-dihydro-6-quinolinyl]propanamide;

10. N-(5-cyclobutyl-1H-pyrazol-3-yl)-2-[2-(4-methoxyphenyl)-4-oxo-1,4-dihydro-6-quinolinyl]propanamide;

11. N-(5-cyclopentyl-1H-pyrazol-3-yl)-2-[2-(4-methoxyphenyl)-4-oxo-1,4-dihydro-6-quinolinyl]propanamide;

12. N-(5-cyclopropyl-1H-pyrazol-3-yl)-2-[2-(4-methoxyphenyl)-4-oxo-4H-thiochromen-6-yl]acetamide;

13. N-(5-cyclobutyl-1H-pyrazol-3-yl)-2-[2-(4-methoxyphenyl)-4-oxo-4H-thiochromen-6-yl]acetamide;

14. N-(5-cyclopentyl-1H-pyrazol-3-yl)-2-[2-(4-methoxyphenyl)-4-oxo-4H-thiochromen-6-yl]acetamide;

15. N-(5-cyclopropyl-1H-pyrazol-3-yl)-2-[8-fluoro-2-(4-methoxyphenyl)4-oxo-4H-chromen-6-yl]acetamide;

16. N-(5-cyclopropyl-1H-pyrazol-3-yl)-2-[5-fluoro-2-(4-methoxyphenyl)-4-oxo-4H-chromen-6-yl]acetamide;

17. N-(5-cyclopropyl-1H-pyrazol-3-yl)-2-[7-fluoro-2-(4-methoxyphenyl)-4-oxo-4H-chromen-6-yl]acetamide;

18. 2-[8-chloro-2-(4-methoxyphenyl)4-oxo-4H-chromen-6-yl]-N-(5-cyclopropyl-1H-pyrazol-3-yl)acetamide;

19. 2-[5-chloro-2-(4-methoxyphenyl)-4-oxo-4H-chromen-6-yl]-N-(5-cyclopropyl-1H-pyrazol-3-yl)acetamide;

20.2-[7-chloro-2-(4-methoxyphenyl)-4-oxo-4H-chromen-6-yl]-N-(5-cyclopropyl-1H-pyrazol-3-yl)acetamide;

21. 2-[8-bromo-2-(4-methoxyphenyl)-4-oxo-4H-chromen-6-yl]-N-(5-cyclopropyl-1H-pyrazol-3-yl)acetamide;

22. 2-[5-bromo-2-(4-methoxyphenyl)-4-oxo-4H-chromen-6-yl]-N-(5-cyclopropyl-1H-pyrazol-3-yl)acetamide;

23. 2-[7-bromo-2-(4-methoxyphenyl)-4-oxo-4H-chromen-6-yl]-N-(5-cyclopropyl-1H-pyrazol-3-yl)acetamide;

24. N-(5-cyclopropyl-1H-pyrazol-3-yl)-2-[5-methoxy-2-(4-methoxyphenyl)-4-oxo-4H-chromen-6-yl]acetamide;

25. N-(5-cyclopropyl-1H-pyrazol-3-yl)-2-[8-methoxy-2-(4-methoxyphenyl)-4-oxo-4H-chromen-6-yl]acetamide;

26. N-(5-cyclopropyl-1H-pyrazol-3-yl)-2-[7-methoxy-2-(4-methoxyphenyl)4-oxo-4H-chromen-6-yl]acetamide;

27. N-(5-cyclopropyl-1H-pyrazol-3-yl)-2-[2-(3-methoxyphenyl)-4-oxo-4H-chromen-6-yl]acetamide;

28. N-(5-cyclopropyl-1H-pyrazol-3-yl)-2-[2-(3-hydroxyphenyl)-4-oxo-4H-chromen-6-yl]acetamide;

29. 2-[2-(3-aminophenyl)-4-oxo-4H-chromen-6-yl]-N-(5-cyclopropyl-1H-pyrazol-3-yl)acetamide;

30. N-(5-cyclopropyl-1H-pyrazol-3-yl)-2-[2-(4-hydroxyphenyl)-4-oxo-4H-chromen-6-yl]acetamide;

31. 2-[2-(4-aminophenyl)-4-oxo-4H-chromen-6-yl]-N-(5-cyclopropyl-1H-pyrazol-3-yl)acetamide;

32. 2-[2-(4-chlorophenyl)-4-oxo-4H-chromen-6-yl]-N-(5-cyclopropyl-1H-pyrazol-3-yl)acetamide;

33. 2-[2-(4-bromophenyl)-4-oxo-4H-chromen-6-yl]-N-(5-cyclopropyl-1H-pyrazol-1-3-yl)acetamide;

34. N-(5-cyclopropyl-1H-pyrazol-3-yl)-2-[2-(4-fluorophenyl)-4-oxo-4H-chromen-6-yl]acetamide;

35. 2-[2-(3-chlorophenyl)4-oxo-4H-chromen-6-yl]-N-(5-cyclopropyl-1H-pyrazol-3-yl)acetamide;

36. 2-[2-(3-bromophenyl)-4-oxo-4H-chromen-6-yl]-N-(5-cyclopropyl-1H-pyrazol-3-yl)acetamide;

37. N-(5-cyclopropyl-1H-pyrazol-3-yl)-2-[2-(3-fluorophenyl)-4-oxo-4H-chromen-6-yl]acetamide;

38. 6-{2-[(5-cyclopropyl-1H-pyrazol-3-yl)amino]-2-oxoethyl}4-oxo-4H-chromene-2-carboxamide;

39. 6-{2-[(5-cyclopropyl-1H-pyrazol-3-yl)amino]-2-oxoethyl}-N-methyl-4-oxo-4H-chromene-2-carboxamide;

40. N-(5-cyclopropyl-1H-pyrazol-3-yl)-2-[4-oxo-2-(1H-pyrrol-2-yl)-4H-chromen-6-yl]acetamide;

41. N-(5-cyclopropyl-1H-pyrazol-3-yl)-2-[4-oxo-2-(1H-pyrazol-5-yl)-4H-chromen-6-yl]acetamide;

42. N-(5-cyclopropyl-1H-pyrazol-3-yl)-2-[2-(1H-imidazol-5-yl)-4-oxo-4H-chromen-6-yl]acetamide;

43. N-(5-cyclopropyl-1H-pyrazol-3-yl)-2-[4-oxo-2-(5-oxo-2-pyrrolidinyl)-4H-chromen-6-yl]acetamide;

44. N-(5-cyclopropyl-1H-pyrazol-3-yl)-2-[2-(2-furyl)-4-oxo-4H-chromen-6-yl]acetamide;

45. N-(5-cyclopropyl-1H-pyrazol-3-yl)-2-[2-(3-furyl)-4-oxo-4H-chromen-6-yl]acetamide;

46. N-(5-cyclopropyl-1H-pyrazol-3-yl)-2-[4-oxo-2-(3-thienyl)-4H-chromen-6-yl]acetamide;

47. N-(5-cyclopropyl-1H-pyrazol-3-yl)-2-[4-oxo-2-(2-thienyl)-4H-chromen-6-yl]acetamide;

48. N-(5-cyclopropyl-1H-pyrazol-3-yl)-2-[4-oxo-2-(2-pyridinyl)-4H-chromen-6-yl]acetamide;

49. N-(5-cyclopropyl-1H-pyrazol-3-yl)-2-[4-oxo-2-(3-pyridinyl)-4H-chromen-6-yl]acetamide;

50. N-(5-cyclopropyl-1H-pyrazol-3-yl)-2-(2-hydroxy-4-oxo-4H-chromen-6-yl)acetamide;

51. 2-(2-amino-4-oxo-4H-chromen-6-yl)-N-(5-cyclopropyl-1H-pyrazol-1-3-yl)acetamide;

52.2-{2-[(aminocarbonyl)amino]-4-oxo-4H-chromen-6-yl}-N-(5-cyclopropyl-1H-pyrazol-3-yl)acetamide;

53. 2-(2-{[amino(imino)methyl]amino}4-oxo-4H-chromen-6-yl)-N-(5-cyclopropyl-1H-pyrazol-3-yl)acetamide;

54. N-(5-cyclopropyl-1H-pyrazol-3-yl)-2-(2-hydroxy-4-oxo-1,4-dihydro-6-quinolinyl)acetamide;

55. 6-{2-[(5-cyclopropyl-1H-pyrazol-3-yl)amino]-2-oxoethyl}-4-oxo-1,4-dihydro-2-quinolinecarboxamide;

56. 6-{2-[(5-cyclopropyl-1H-pyrazol-3-yl)amino]-2-oxoethyl}-N-methyl-4-oxo-1,4-dihydro-2-quinolinecarboxamide;

57. N-(5-cyclopropyl-1H-pyrazol-3-yl)-2-[4-oxo-2-(1H-pyrrol-2-yl)-1,4-dihydro-6-quinolinyl]acetamide;

58. N-(5-cyclopropyl-1H-pyrazol-3-yl)-2-[4-oxo-2-(1H-pyrazol-5-yl)-1,4-dihydro-6-quinolinyl]acetamide;

59. N-(5-cyclopropyl-1H-pyrazol-3-yl)-2-[2-(1H-imidazol-5-yl)-4-oxo-1,4-dihydro-6-quinolinyl]acetamide;

60. N-(5-cyclopropyl-1H-pyrazol-3-yl)-2-[2-(4-methoxyphenyl)-1-methyl-4-oxo-1,4-dihydro-6-quinolinyl]acetamide;

61. N-(5-cyclopropyl-1H-pyrazol-3-yl)-2-[1-(2-hydroxyethyl)-2-(4-methoxyphenyl)4-oxo-1,4-dihydro-6-quinolinyl]acetamide;

62. 2-[1-(2-aminoethyl)-2-(4-methoxyphenyl)-4-oxo-1,4-dihydro-6-quinolinyl]-N-(5-cyclopropyl-1H-pyrazol-3-yl)acetamide;

63. N-(5-cyclopropyl-1H-pyrazol-3-yl)-2-[2-(4-methoxyphenyl)-3-methyl-4-oxo-4H-chromen-6-yl]acetamide;

64. N-(5-cyclopropyl-1H-pyrazol-3-yl)-2-(4-oxo-4H-chromen-6-yl)acetamide;

65. N-(5-cyclopropyl-1H-pyrazol-3-yl)-2-(4-oxo-1,4-dihydro-6-quinolinyl)acetamide;

66. N-(5-cyclopropyl-1H-pyrazol-3-yl)-2-(4-oxo-4H-thiochromen-6-yl)acetamide;

67. N-(5-cyclopropyl-1H-pyrazol-3-yl)-2-[3-hydroxy-2-(4-methoxyphenyl)-4-oxo-4H-chromen-6-yl]acetamide;

68. N-(5-cyclopropyl-1H-pyrazol-3-yl)-2-[3-methoxy-2-(4-methoxyphenyl)4-oxo-4H-chromen-6-yl]acetamide;

69. 2-[3-(benzyloxy)-2-(4-methoxyphenyl)-4-oxo-4H-chromen-6-yl]-N-(5-cyclopropyl-1H-pyrazol-3-yl)acetamide;

70. 6-{2-[(5-cyclopropyl-1H-pyrazol-3-yl)amino]-2-oxoethyl}-2-(4-methoxyphenyl)-4-oxo-4H-chromene-3-carboxamide;

71. 2-(3-acetyl-4-oxo-4H-chromen-6-yl)-N-(5-cyclopropyl-1H-pyrazol-3-yl)acetamide;

72. 2-(3-benzoyl-4-oxo-4H-chromen-6-yl)-N-(5-cyclopropyl-1H-pyrazol-3-yl)acetamide.

The compounds of formula (I) and the salts thereof, object of the invention, may be obtained by a process comprising:

a) reacting the compounds of formula (m) or the regioisomers of formula (IIa)

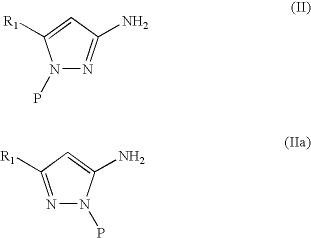

wherein R₁ is as above defined and P represents a suitable nitrogen-pyrazole protecting group, with the compounds of formula

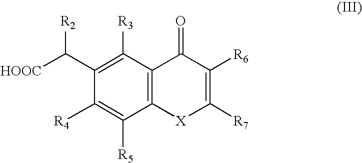

wherein R₂, R₃, R₄, R₅, R₆, R₇ and X are as above defined, thus obtaining the compounds of formula (IV) or (IVa)

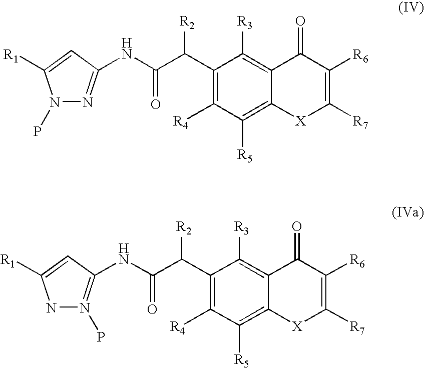

b) and deprotecting the compounds of formula (V) or (IVa) so as to obtain the derivatives of formula (I) and, if desired, converting them into pharmaceutically acceptable salts thereof.

The above process is an analogy process which can be carried out according to well known methods.

According to step a) of the process, the reaction between the compounds of formula (II) or (IIa) with the compounds of formula (III) can be carried out in the presence of a coupling agent, for instance a carbodiimide such as 1,3-dicyclohexylcarbodiimide, 1,3-diisopropylcarbodiimide or 1-(3-dinethylaminopropyl)-3-ethylcarbodiimide, optionally in the presence of a tertiary base such as triethylamine, N-methylmorpholine, N,N-diisopropylethylamine or pyridine.

The reaction may occur in a suitable solvent such as, for example, dichloromethane, chloroform, tetrahydrofuran, diethylether, 1,4-dioxane, acetonitrile, toluene or N,N-dimethylformaride, at a temperature ranging from about −10° C. to reflux and for a suitable time, for instance from about 30 minutes to about 96 hours.

Alternatively, step a) of the process can also be carried out by a mixed anhydride method, that is by using an alkyl chloroformate such as ethyl, isobutyl or isopropyl chloroformate, in the presence of a tertiary base such as triethylamine, N-methylmorpholine, N,N-diisopropylethylamine or pyridine, in a suitable solvent such as toluene, dichloromethane, chloroform, tetrahydrofuran, acetonitrile, diethylether, 1,4-dioxane or N,N-dimethylformamide, at a temperature ranging from about −30° C. to room temperature.

As far as the compounds of formula (II) or (IIa) are concerned, suitable P groups are those conventionally used to protect pyrazole-nitrogen atoms. Preferably, for both compounds (II) and (IIa), P represents a tert-butoxycarbonyl (BOC) group.

In step b) of the process, the compounds of formula (IV) or (IVa) are converted into the desired derivatives of formula (I) by deprotecting the pyrazole-nitrogen atom according to conventional methods.

As an example, deprotection from BOC may occur under acidic conditions, for instance in the presence of trifluoroacetic, formic or hydrochloric acid, in a suitable solvent such as dichloromethane, and at a temperature ranging from about 0° C. to room temperature.

The compounds of formula (I) or (IIa) are known or may be prepared according to known methods by starting from the corresponding deprotected pyrazole derivatives of formula

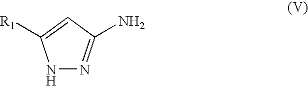

wherein R₁ is as above defined.

For a reference to the preparation of the compounds of formula (II) see, for instance, the aforementioned international patent application WO 01/12189.

When preparing the compounds of formula (IIa), the compounds of formula (V) are protected, for instance as BOC derivatives, through reaction with tert-butoxycarbonyl anhydride in the presence of a suitable solvent, for instance a dichloromethane/water admixture, and of a base such as sodium hydroxide, carbonate or bicarbonate.

Alternatively, this same reaction may be carried out in toluene, tetrahydrofuran or 1,4-dioxane in the presence of a base, for instance triethylamine or N,N-diisopropylethylamine.

Also the compounds of formula (III) are known or may be prepared according to known methods.

As an example, the compounds of formula (III) can be prepared by a process comprising:

a) reacting the compounds of formula

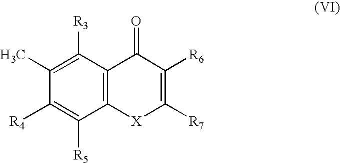

wherein R₃, R₄, R₅, R₆, R₇ and X are as above defined, with N-bromosuccinimide in the presence of a suitable peroxide, to give the corresponding compounds of formula

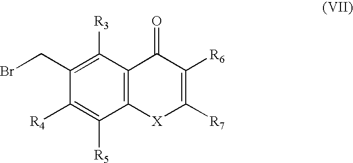

b) reacting the compounds of formula (VII) with an alkaline cyanide so as to obtain the corresponding ciano derivatives which, hydrolyzed according to conventional techniques, for instance under acidic conditions, give the corresponding compounds of formula (III) wherein R₂ is a hydrogen atom and, if desired,

c) alkylating them, in the presence of a base, with a suitable halide derivative of formula

R₂−Hal (VIII)

wherein Hal is a halogen atom, for instance iodine, so as to obtain the compounds of formula (III) wherein R₂ is an alkyl or alkenyl group as above defined.

The reaction of steps a) and b) for preparing the compounds of formula (III) wherein R₂ is a hydrogen atom may be carried out according to known methods, for instance as reported in J. Indian Chem. Soc. (1973), 295-298.

The reaction of the compounds of formula (VI) with N-bromosuccinimide is carried out in the presence of a peroxide, for instance benzoyl peroxide, in a suitable solvent such as dry benzene or toluene at refluxing temperature.

The compounds of formula (VII) are then easily converted into the corresponding carboxy derivatives of formula (III) wherein R₂ is a hydrogen atom by first reacting them with an alkaline cyanide, for instance potassium cyanide, in the presence of a suitable solvent such as ethanol, at refluxing temperature. The cyanomethyl derivatives thus prepared are then hydrolyzed to the corresponding carboxy derivatives (III), for instance with sulfuric acid.

The alkylation reaction of the compounds of formula (III) wherein R₂ is hydrogen to yield the corresponding compounds of formula (III) wherein R₂ is alkyl or alkenyl, can be carried out in the presence of a base such as sodium hydride, lithium diisopropylamine, potassium tert-butylate or carbonate, in a suitable solvent such as tetrahydrofuran, N,N-dimethylformamide, dimethoxyethane or 1,4-dioxane, at a temperature ranging from about −78° C. to reflux.

Also the compounds of formula (VI) are known or can be prepared according to known methods.

As an example, the compounds of formula (VI) wherein X is an oxygen atom may be prepared by a process comprising:

a) reacting the compounds of formula

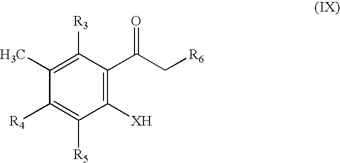

wherein R₃, R₄, R₅ and R₆ are as above defined and X is oxygen; with the compounds of formula

R₇COOMe (X)

wherein R₇ is as above defined; thus obtaining the compounds of formula

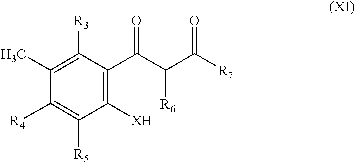

b) and cyclising the compounds of formula (XI) in acidic medium, thus obtaining the compounds of formula (VI).

The reaction of the compounds of formula (IX) with the compounds of formula (X), as per step a), can be carried in the presence of a base such as sodium hydride, in a suitable solvent such as 1,4-dioxane, tetrahydrofuran or diethylether, at a temperature ranging from room temperature to reflux.

The reaction of the compounds of formula (XI) to produce the compounds of formula (VI), according to the cyclisation step b) of the process, can be carried out in a suitable solvent such as ethanol or acetic acid, in the presence of concentrated hydrochloric acid or with formic acid at refluxing temperature.

Alternatively, the compounds of formula (VI) wherein R₃, R₄, R₅, R₆ and R₇ are as above defined and X is oxygen or a group —N(R₈)— wherein R₈ is as above defined, may be prepared according to a process which comprises:

a) reacting the compounds of formula (DI) wherein R₃, R₄, R₅ and R₆ are as above defined and X is oxygen or —N(R₈)—, with the compounds of formula

R₇COCl (XII)

wherein R₇ is as above defined, thus obtaining the compounds of formula

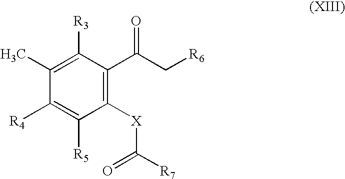

b) and by carrying out the Baker-Venkataraman transformation of (XIII), under basic conditions, so as to obtain the compounds of formula (XI) which are subsequently cyclised to the derivatives of formula (VI) as above defined.

The reaction of step a) between the compounds of formula (IX) and the compounds of formula (XII) can be carried out, for instance, in dry pyridine, at room temperature and for a time ranging from about 1 hour to about 20 hours.

The reaction of the compounds of formula (XIII) according to the Baker-Venkataraman transposition, is carried out in the presence of a base, for instance potassium carbonate, in a suitable solvent such as isopropanol, at refluxing temperature.

Alternatively, the compounds of formula (VI) wherein R₃, R₄, R₅, R₆ and R₇ are as above defined and X is oxygen or a group —N(R₈)— wherein R₈ is as above defined, may be prepared according to a process comprising:

a) reacting, under basic conditions and in the presence of palladium Pd(0) and carbon monoxide, the compounds of formula

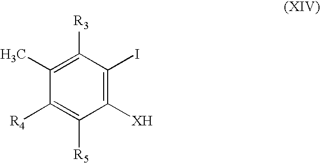

with the compounds of formula (XV) below wherein R₆ and R₇ are as above defined

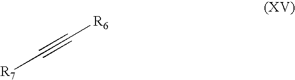

The reaction between the compounds of formula (XIV) and the compounds of formula (XV) can be carried out in the presence of gaseous CO (5-20 atm; 5-20·10⁵ Pa) and of a Pd(0) catalyst, for instance originating from PdCl₂(PPh₃)₂-thiourea complex, PdCl₂(dppf) [wherein dppf stands for 1,1′-bis(diphenylphosphino)ferrocene] and PdCl₂(CH₃CN)₂, in the presence of 1,8-diazabicyclo[5.4.0]undec-7-ene (DBU), diethylamine, triethylamine, piperidine or morpholine, in a suitable solvent such as benzene or hexamethylphosphoramide (HMPA), at a temperature ranging from room temperature to about 120° C.

Alternatively, the compounds of formula (VI) wherein R₃, R₄, R₅, R₆ and R₇ are as above defined and X is a sulfur atom can be prepared by a process comprising reacting, under acidic conditions, the compounds of formula

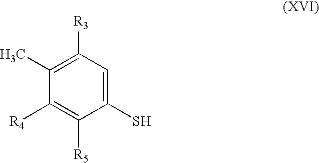

with the compounds of formula (III below wherein R₆ and R₇ are as above defined and R′ is an alkyl group

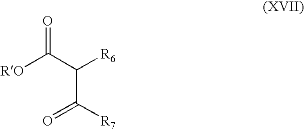

The reaction between the compounds of formula (XVI) with the compounds of formula (XVII) can be carried out in a suitable solvent such acetonitrile in the presence of an acid such as sulfuric, polyphosphoric or chlorosulfonic acid, at a temperature ranging from room temperature to reflux.

As will be readily appreciated, if the compounds of formula (I), prepared according to any one of the processes described above, are obtained as an admixture of isomers, their separation into the single isomers of formula (I), according to conventional techniques, is within the scope of the present invention. Conventional techniques for racemate resolution include, for instance, partitioned crystallization of diastereoisomeric salt derivatives or preparative chiral HPLC.

Likewise, the optional conversion of a compound of formula (I) into another compound of formula (I), the optional salification of a compound of formula (I) or the conversion of a salt thereof into the free compound, all carried out according to conventional methods, are still within the scope of the present invention.

When preparing the compounds of formula (I), optional functional groups within both the starting materials or the intermediates thereof which could give rise to unwanted side reactions are preferably protected according to conventional techniques. Likewise, the conversion of these protected compounds into the free deprotected derivatives may be carried out according to well-known procedures.

All of the compounds of formula (V), (VIII), (IX), (X), (XII), (XIV), (XVI) and (XVII) are known or may be easily prepared according to known methods.

Finally, it is clear to the man skilled in the art that the compounds of formula (I) of the invention can be also prepared by performing the above described reactions in a combinatorial fashion, according to conventional methods.

As an example, the compounds of formula (III) may be supported onto resin particles and further reacted with a variety of compounds of formula (II) or (IIa), so as to obtain several different compounds of formula (I), according to solid phase synthesis (SPS) techniques applied to combinatorial chemistry methods. These derivatives, in their turn, are then conveniently converted into the derivatives of formula (I) of the invention.

Pharmacology

The compounds of formula (I) are active as cdk/cyclin inhibitors as they gave positive results when test according to the following procedure.

The inhibiting activity of putative cdk/cyclin inhibitors and the potency of selected compounds was determined through a method of assay based on the use of the MultiScreen-PH 96 well plate (Mllipore), in which phosphocellulose filter paper was placed at each well bottom allowing binding of positive charged substrate after a washing/filtration step.

When a radioactivity labeled phosphate moiety was transferred by the ser/threo kinase to the filter-bound histone, light emitted was measured in a scintillation counter.

The Inhibition Assay of cdk2 Cyclin A Activity Performed According to the Following Protocol:

Kinase reaction: 1.5 μM histone H1 substrate, 25 μM ATP (0.5 uCi P33g-ATP), 100 ng Cyclin A/cdk2 complex, 10 μM inhibitor in a final volume of 100 μl buffer (TRIS HCl 10 mM pH 7.5, MgCl2 10 mM, 7.5 mM DTT) were added to each well of a 96 U bottom well plate. After 10 min at 37 C. incubation, reaction was stopped by 20 μl EDTA 120 mM.

Capture: 100 μl were transferred from each well MultiScreen plate, to allow substrate binding phosphocellulose filter. Plates were then washed 3 times with 150 μl/well PBS Ca++/Mg++ free and filtered by MultiScreen filtration system.

Detections: filters were allowed to dry at 37° C., then 100 μl/well scintillant were added and 33P labeled histone H1 was detected by radioactivity counting in the Top-Count instrument.

Results: data were analyzed and expressed as % inhibition referred to total activity of enzyme (=100%).

All compounds showing inhibition >50% were further analyzed in order to study and define the kinetic-profile of the inhibitor via Ki calculation.

The protocol used was the same described above, except for ATP and substrate concentrations. Either the concentrate of ATP and histone H1 substrate were varied: 4, 8, 12, 24, 48 μM for ATP (containing proportionally diluted P33g-ATP) and 0.4, 0.8, 1.2, 2.4, 4.8 μM for histone were used in absence and presence of two different, properly chosen inhibitor concentrations.

Experimental data were analyzed by the computer program “SigmaPlot” for Ki determination, using a random bireactant system equation:

$$v = \frac{\begin{matrix}{V}_{max} & & ( A ) ( B ) \\ & & a K A K B\end{matrix}}{\begin{matrix}1 + ( A ) + & ( B ) + & ( A ) ( B ) \\ K A & K B & aKAKB\end{matrix}}$$

where A=ATP and B=histone H1.

In addition, the inhibiting activity of putative cdk/cyclin inhibitors and the potency of selected compounds was determined using a method of assay based on the use of a SPA (Scintillation Proximity Assay) 96 well plate assay. The assay is based on the ability of streptavidin-coated SPA beads to capture a biotinylated peptide derived from a phosphorylation site of histone.

When a radioactivity labeled phosphate moiety was transferred by the ser/threo kinase to the biotinylated histone peptide, light emitted was measured in scintillation counter.

The inhibition assay of cdk5/p25 activity was performed according to the following protocol;

Kinase reaction: 1.0 μM biotinylated histone peptide substrate, 0.25 uCi P33g-ATP, 4 nM cdk2/p25 complex, 0-100 μM] inhibitor in a final volume of 100 μl buffer (Hepes 20 mM pH 7.5, MgCl2 15 mM, 1 mM DTT) were added to each well of a 96 U bottom well plate. After 20 min at 37° C. incubation, the reaction was stopped by the addition of 500 ug SPA beads in phosphate-buffered saline containing 0.1% Triton X-100, 50 M ATP and 5 mM EDTA. The beads were allowed to settle, and the radioactivity incorporated in the 33P-labelled peptide was detected in a Top Count scintillation counter.

Results: Data were analyzed and expressed as % Inhibition using the formula:

100×(1−(Unknown−Bkgd)/(Enz. Control−Bkgd))

IC50 values were calculated using a variation of the four parameter logistics equation:

Y=100/[1+10\hat{ }{(LogEC50−X)*Slope}]

Where X=log(uM) and Y=% Inhibition.

The compounds of formula (I) are therefore useful to restrict the unregulated proliferation of tumor cells, hence in therapy in the treatment of various tumors such as, for instance, carcinomas, e.g., mammary carcinoma, carcinoma, bladder carcinoma, colon carcinoma, ovary endometrial tumors, sarcomas, e.g., soft tissue and bone sarcomas, and the hematological malignancies such as, e.g., leukemias.

In addition, the compounds of formula (I) are also useful in the treatment of other cell proliferative disorders such as psoriasis, vascular smooth cell proliferation associated with atherosclerosis and post-surgical stenosis a restenosis, and in the treatment of Alzheimer's disease.

The compounds of the present invention can be administered either as single agents or, alternatively, in combination with known anticancer treatments such as radiation therapy or chemotherapy regimen in combination with cytostatic or cytotoxic agents, antibiotic-type agents, alkylating agents, antimetabolite agents, hormonal agents, immunological agents, interferon-type agents, cyclooxygenase inhibitors (e.g. COX-2 inhibitors), metallomatrixprotease inhibitors, telomerase inhibitors, tyrosine kinase inhibitors, anti-growth factor receptor agents, anti-HER agents, anti-EGFR agents, anti-angiogenesis agents (e.g. angiogenesis inhibitors), farnesyl transferase inhibitors, ras-raf signal transduction pathway inhibitors, cell cycle inhibitors, other cdks inhibitors, tubulin binding agents, topoisomerase I inhibitors, topoisomerase II inhibitors, and the like.

As an example, the compounds of the invention can be administered in combination with one or more chemotherapeutic agents such as, for instance, taxane, taxane derivatives, encapsulated taxanes, CPT-11, SN-38, camptothecin derivatives, anthracycline glycosides, e.g., doxorubicin, idarubicin, epirubicin, etoposide, navelbine, vinblastine, carboplatin, cisplatin, estramustine, celecoxib, Sugen SU-5416, Sugen SU-6668, Herceptin, and the like, optionally within liposomal formulations thereof.

If formulated as a fixed dose, such combination products employ the compounds of this invention within the dosage range described below and the other pharmaceutically active agent within the approved dosage range.

Compounds of formula (I) may be used sequentially with known anticancer agents when a combination formulation is inappropriate.

The compounds of formula (I) of the present invention, suitable for administration to a mammal, e.g., to humans, can be administered by the usual routes and the dosage, level depends upon the age, weight, conditions of patient and the administration route.

For example, a suitable dosage adopted for oral administration of a compound of formula (I) may range from about 10 to about 500 mg per dose, from 1 to 5 times daily. The compounds of the invention can be administered in a variety of dosage forms, e.g., orally, in the form tablets, capsules, sugar or film coated tablets, liquid solutions or suspensions; rectally in the form suppositories; parenterally, e.g., intramuscularly, or intravenous and/or intrathecal and/or intraspinal injection or infusion.

The present invention also includes pharmaceutical compositions comprising a compound of formula (I) or a pharmaceutically acceptable salt thereof in association with a pharmaceutically acceptable excipient, which may be a carrier or a diluent.

The pharmaceutical compositions containing the compounds of the invention are usually prepared following convention methods and are administered in a pharmaceutically suitable form.

For example, the solid oral forms may contain, together with the active compound, diluents, e.g., lactose, dextrose saccharose, sucrose, cellulose, corn starch or potato starch; lubricants, e.g., silica, talc, stearic acid, magnesium or calcium stearate, and/or polyethylene glycols; binding agents, e.g., starches, arabic gum, gelatine, methylcellulose, carboxymethylcellulose or polyvinyl pyrrolidone; disintegrating agents, e.g., a starch, alginic acid, alginates or sodium starch glycolate; effervescing mixtures; dyestuffs; sweeteners; wetting agents such as lecithin, polysorbates, laurylsulphates; and, in general, non-toxic and pharmacologically inactive substances used in pharmaceutical formulations. These pharmaceutical preparations may be manufactured in known manner, for example, by means of mixing, granulating, tabletting, sugar-coating, or film-coating processes.

The liquid dispersions for oral administration may be, e.g., syrups, emulsions and suspensions.

The syrups may contain as carrier, for example, saccharose or saccharose with glycerine and/or mannitol and sorbitol.

The suspensions and the emulsions may contain as carrier, for example, a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g., sterile water, olive oil, ethyl oleate, glycols, e.g., propylene glycol, and, if desired, a suitable amount of lidocaine hydrochloride.

The solutions for intravenous injections or infusions may contain as a carrier, for example, sterile water or preferably they may be in the form of sterile, aqueous isotonic saline solutions or they may contain as a carrier propylene glycol,

The suppositories may contain together with the active compound a pharmaceutically acceptable carrier, e.g., cocoa butter, polyethylene glycol, a polyoxyethylene sorbitan fatty acid ester surfactant or lecithin.

### EXAMPLES

Having generally described this invention, a further understanding can be obtained by reference to certain specific examples which are provided herein for purposes of illustration only and are not intended to be limiting unless otherwise specified.

### Example 1

Tert-butyl-5-amino-3-cyclopropyl-1H-pyrazole-1-carboxylate

0.81 g (6.6 mmol) of 5-cyclopropyl-3-amino-pyrazole were dissolved in a mixture of 20 ml of aqueous sodium hydroxide and 20 ml of CH₂Cl₂. 2.8 g (13.2 mmol) of tert-butoxycarbonylanhydride were added and the solution was maintained at room temperature under stirring overnight. The organic layer was separated, washed with water, dried over Na₂SO₄ and evaporated. The title compound was crystallized from n-hexane (1 g, 71% yield).

Following the same method, but employing 5-cyclobutyl-3-amino-pyrazole and 5-cyclopentyl-3-amino-pyrazole, tert-butyl-5-amino-3-cyclobutyl-1H-pyrazole-1-carboxylate and tert-butyl-5-amino-3-cyclopentyl-1H-pyrazole-1-carboxylate can be respectively obtained.

### Example 2

N-(3-cyclopropyl-1H-pyrazol-5-yl)-2-[2-(4-methoxyphenyl) 4-oxo-4H-chromen-6-yl]acetamide

2.79 g (9 mmol) of 2-(4-methoxyphenyl)-4-oxo-4H-chromen-6-yl]acetic acid were suspended in 30 ml of CH₂Cl₂ and 1.55 ml (9 mmol) of N,N-diisopropylethylamine and 1.7 g (9 mmol) of N-ethyl-N′-diisopropylcarbodiimide were added, at 0° C. under stirring. After 30 minutes at the same temperature, a solution of 1 g (4.5 mmol) of tert-butyl-5-amino-3-cyclopropyl 1H-pyrazole-1-carboxylate in 10 ml of CH₂Cl₂ was added dropwise. After 12 hours at room temperature the mixture was washed with a saturated sodium hydrogenocarbonate solution and then with water. The organic layer was dried over anhydrous Na₂SO₄ and evaporated to give 1.38 g (60% yield) of tert-butyl-3-cyclopropyl-5-{3-[2-(4-methoxyphenyl)4-oxo-4H-chromen-6-yl]-2-oxopropyl}-1H-pyrazole-1-carboxylate. This intermediate, without further purification, was redissolved with 30 ml of a mixture 9/1 of CH₂Cl₂/TFA and the resulting solution was maintained at room temperature for 3 hours. The solvent was then evaporated, CH₂Cl₂ added to the residue and the mixture washed with a saturated NaHCO₃ solution. The organic layer was dried over anhydrous Na₂SO₄ and concentrated under vacuum. 0.99 g (90% yield) of the title compound crystallized from n-hexane were thus obtained.

By working in analogous way and by using the proper starting material, the following compounds can be prepared:

N-(5-cyclobutyl-1H-pyrazol-3-yl)-2-[2-(4-methoxyphenyl)-4-oxo-4H-chromen-6-yl]acetamide;

N-(5-cyclopentyl-1H-pyrazol-3-yl)-2-[2-(4-methoxyphenyl)-oxo-4H-chromen-6-yl]acetamide;

N-(5-cyclopropyl-1H-pyrazol-3-yl)-2-[2-(4-methoxyphenyl)-4-oxo-1,4-dihydro-6-quinolinyl]acetamide;

N-(5-cyclobutyl-1H-pyrazol-3-yl)-2-[2-(4-methoxyphenyl)-4-oxo-1,4-dihydro-6-quinolinyl]acetamide;

N-(5-cyclopentyl-1H-pyrazol-3-yl)-2-[2-(4-methoxyphenyl)-4-oxo-1,4-dihydro-6-quinolinyl]acetamide;

N-(5-cyclobutyl-1H-pyrazol-3-yl)-2-[2-(4-methoxyphenyl)-4-oxo-4H-chromen-6-yl]propanamide;

N-(5-cyclopentyl-1H-pyrazol-3-yl)-2-[2-(4-methoxyphenyl)-4-oxo-4H-chromen-6-yl]propanamide;

N-(5-cyclopropyl-1H-pyrazol-3-yl)-2-[2-(4-methoxyphenyl)-4-oxo-1,4-dihydro-6-quinolinyl]propanamide;

N-(5-cyclobutyl-1H-pyrazol-3-yl)-2-[2-(4-methoxyphenyl)4-oxo-1,4-dihydro-6-quinolinyl]propanamide;

N-(5-cyclopentyl-1H-pyrazol-3-yl)-2-[2-(4-methoxyphenyl)4-oxo-1,4 dihydro-6-quinolinyl]propanamide;

N-(5-cyclopropyl-1H-pyrazol-3-yl)-2-[2-(4-methoxyphenyl)-4-oxo-4H-thiochromen-6-yl]acetamide;

N-(5-cyclobutyl-1H-pyrazol-3-yl)-2-[2-(4-methoxyphenyl)-4-oxo-4H-thiochromen-6-yl]acetamide;

N-(5-cyclopentyl-1H-pyrazol-3-yl)-2-[2-(4-methoxyphenyl)-4-oxo-4H-thiochromen-6-yl]acetamide;

N-(5-cyclopropyl-1H-pyrazol-3-yl)-2-[8-fluoro-2-(4-methoxyphenyl)-4-oxo-4H-chromen-6-yl]acetamide;

N-(5-cyclopropyl-1H-pyrazol-3-yl)-2-[5-fluoro-2-(4-methoxyphenyl)-4-oxo-4H-chromen-6-yl]acetamide;

N-(5-cyclopropyl-1H-pyrazol-3-yl)-2-[7-fluoro-2-(4-methoxyphenyl)-4-oxo-4H-chromen-6-yl]acetamide;

2-[8-chloro-2-(4-methoxyphenyl)-4-oxo-4H-chromen-6-yl]-N-(5-cyclopropyl-1H-pyrazol-3-yl)acetamide;

2-[5-chloro-2-(4-methoxyphenyl)-4-oxo-4H-chromen-6-yl]-N-(5-cyclopropyl-1H-pyrazol-3-yl)acetamide;

2-[7-chloro-2-(4-methoxyphenyl)-4-oxo-4H-chromen-6-yl]-N-(5-cyclopropyl-1H-pyrazol-3-yl)acetamide;

2-[8-bromo-2-(4-methoxyphenyl)-4-oxo-4H-chromen-6-yl]-N-(5-cyclopropyl-1H-pyrazol-3-yl)acetamide;

2-[5-bromo-2-(4-methoxyphenyl)-4-oxo-4H-chromen-6-yl]-N-(5-cyclopropyl-1H-pyrazol-3-yl)acetamide;

2-[7-bromo-2-(4-methoxyphenyl)-4-oxo-4H-chromen-6-yl]-N-(5-cyclopropyl-1H-pyrazol-3-yl)acetamide;

N-(5-cyclopropyl-1H-pyrazol-3-yl)-2-[5-methoxy-2-(4-methoxyphenyl)-4-oxo-4H-chromen-6-yl]acetamide;

N-(5-cyclopropyl-1H-pyrazol-3-yl)-2-[8-methoxy-2-(4-methoxyphenyl)4-oxo-4H-chromen-6-yl]acetamide;

N-(5-cyclopropyl-1H-pyrazol-3-yl)-2-[7-methoxy-2-(4-methoxyphenyl)4-oxo-4H-chromen-6-yl]acetamide;

N-(5-cyclopropyl-1H-pyrazol-3-yl)-2-[2-(3-methoxyphenyl)-4-oxo-4H-chromen-6-yl]acetamide;

N-(5-cyclopropyl-1H-pyrazol-3-yl)-2-[2-(3-hydroxyphenyl)4-oxo-4H-chromen-6-yl]acetamide;

2-[2-(3-aminophenyl)4-oxo-4H-chromen-6-yl]-N-(5-cyclopropyl-1H-pyrazol-3-yl)acetamide;

N-(5-cyclopropyl-1H-pyrazol-3-yl)-2-[2-(4-hydroxyphenyl)-4-oxo-4H-chromen-6-yl]acetamide;

2-[2-(4-aminophenyl)-4-oxo-4H-chromen-6-yl]-N-(5-cyclopropyl-1H-pyrazol-3-yl)acetamide;

2-[2-(4-chlorophenyl)-4-oxo-4H-chromen-6-yl]-N-(5-cyclopropyl-1H-pyrazol-3-yl)acetamide;

2-[2-(4-bromophenyl)-4-oxo-4H-chromen-6-yl]-N-(5-cyclopropyl-1H-pyrazol-3-yl)acetamide;

N-(5-cyclopropyl-1H-pyrazol-3-yl)-2-[2-(4-fluorophenyl)-4-oxo-4H-chromen-6-yl]acetamide;

2-[2-(3-chlorophenyl)-4-oxo-4H-chromen-6-yl]-N-(5-cyclopropyl-1H-pyrazol-3-yl)acetamide;

2-[2-(3-bromophenyl)-4-oxo-4H-chromen-6-yl]-N-(5-cyclopropyl-1H-pyrazol-3-yl)acetamide;

N-(5-cyclopropyl-1H-pyrazol-3-yl)-2-[2-(3-fluorophenyl)-4-oxo-4H-chromen-6-yl]acetamide;

6-{2-[(5-cyclopropyl-1H-pyrazol-3-yl)amino]-2-oxoethyl}-4-oxo-4H-chromene-2-carboxamide;

6-{2-[(5-cyclopropyl-1H-pyrazol-3-yl)amino]-2-oxoethyl}-N-methyl-4-oxo-4H-chromene-2-carboxamide;

N-(5-cyclopropyl-1H-pyrazol-3-yl)-2-[4-oxo-2-(1H-pyrrol-2-yl)-4H-chromen-6-yl]acetamide;

N-(5-cyclopropyl-1H-pyrazol-3-yl)-2-[4-oxo-2-(1H-pyrazol-5-yl)-4H-chromen-6-yl]acetamide;

N-(5-cyclopropyl-1H-pyrazol-3-yl)-2-[2-(1H-imidazol-5-yl)-4-oxo-4H-chromen-6-yl]acetamide;

N-(5-cyclopropyl-1H-pyrazol-3-yl)-2-[4-oxo-2-(5-oxo-2-pyrrolidinyl)-4H-chromen-6-yl]acetamide;

N-(5-cyclopropyl-1H-pyrazol-3-yl)-2-[2-(2-furyl)-4-oxo-4H-chromen-6-yl]acetamide;

N-(5-cyclopropyl-1H-pyrazol-3-yl)-2-[2-(3-furyl)4-oxo-4H-chromen-6-yl]acetamide;

N-(5-cyclopropyl-1H-pyrazol-3-yl)-2-[4-oxo-2-(3-thienyl)-4H-chromen-6-yl]acetamide;

N-(5-cyclopropyl-1H-pyrazol-3-yl)-2-[4-oxo-2-(2-thienyl)-4H-chromen-6-yl]acetamide;

N-(5-cyclopropyl-1H-pyrazol-3-yl)-2-[4-oxo-2-(2-pyridinyl)-4H-chromen-6-yl]acetamide;

N-(5-cyclopropyl-1H-pyrazol-3-yl)-2-[4-oxo-2-(3-pyridinyl)-4H-chromen-6-yl]acetamide;

N-(5-cyclopropyl-1H-pyrazol-3-yl)-2-(2-hydroxy-4-oxo-4H-chromen-6-yl)acetamide;

2-(2-amino-4-oxo-4H-chromen-6-yl)-N-(5-cyclopropyl-1H-pyrazol-3-yl)acetamide;

2-{2-[(aminocarbonyl)amino]-4-oxo-4H-chromen-6-yl}-N-(5-cyclopropyl-1H-pyrazol-3-yl)acetamide;

2-(2-{[amino(imino)methyl]amino})-4-oxo-4H-chromen-6-yl)-N-(5-cyclopropyl-1H-pyrazol-3-yl)acetamide;

N-(5-cyclopropyl-1H-pyrazol-3-yl)-2-(2-hydroxy-4-oxo-1,4-dihydro-6-quinolinyl)acetamide;

6-{2-[(5-cyclopropyl-1H-pyrazol-3-yl)amino]-2-oxoethyl}-4-oxo-1,4-dihydro-2-quinolinecarboxamide;

6-{2-[(5-cyclopropyl-1H-pyrazol-3-yl)amino]-2-oxoethyl}-N-methyl-4-oxo-1,4-dihydro-2-quinolinecarboxamide;

N-(5-cyclopropyl-1H-pyrazol-3-yl)-2-[4-oxo-2-(1H-pyrrol-2-yl)-1,4-dihydro-6-quinolinyl]acetamide;

N-(5-cyclopropyl-1H-pyrazol-3-yl)-2-[4-oxo-2-(1H-pyrazol-5-yl)-1,4-dihydro-6-quinolinyl]acetamide;

N-(5-cyclopropyl-1H-pyrazol-3-yl)-2-[2-(1H-imidazol-5-yl)-4-oxo-1,4 dihydro-6-quinolinyl]acetamide;

N-(5-cyclopropyl-1H-pyrazol-3-yl)-2-[2-(4-methoxyphenyl)-1-methyl-4-oxo-1,4-dihydro-6-quinolinyl]acetamide;

N-(5-cyclopropyl-1H-pyrazol-3-yl)-2-[1-(2-hydroxyethyl)-2-(4-methoxyphenyl)-4-oxo-1,4-dihydro-6-quinolinyl]acetamide;

2-[1-(2-aminoethyl)-2-(4-methoxyphenyl)-4-oxo-1,4-dihydro-6-quinolinyl]-N-(5-cyclopropyl-1H-pyrazol-3-yl)acetamide;

N-(5-cyclopropyl-1H-pyrazol-3-yl)-2-[2-(4-methoxyphenyl)-3-methyl-4-oxo-4H-chromen-6-yl]acetamide;

N-(5-cyclopropyl-1H-pyrazol-3-yl)-2-(4-oxo-4H-chromen-6-yl)acetamide;

N-(5-cyclopropyl-1H-pyrazol-3-yl)-2-(4-oxo-1,4-dihydro-6-quinolinyl)acetamide;

N-(5-cyclopropyl-1H-pyrazol-3-yl)-2-(4-oxo-4H-thiochromen-6-yl)acetamide;

N-(5-cyclopropyl-1H-pyrazol-3-yl)-2-[3-hydroxy-2-(4-methoxyphenyl)4-oxo-4H-chromen-6-yl]acetamide;

N-(5-cyclopropyl-1H-pyrazol-3-yl)-2-[3-methoxy-2-(4-methoxyphenyl)-4-oxo-4H-chromen-6-yl]acetamide;

2-[3-(benzyloxy)-2-(4-methoxyphenyl)-4-oxo-4H-chromen-6-yl]-N-(5-cyclopropyl-1H-pyrazol-3-yl)acetamide;

6-{2-[(5-cyclopropyl-1H-pyrazol-3-yl)amino]-2-oxoethyl}-2-(4-methoxyphenyl)-4-oxo-4H-chromene-3-carboxamide;

2-(3-acetyl-4-oxo-4H-chromen-6-yl)-N-(5-cyclopropyl-1H-pyrazol-3-yl)acetamide;

2-(3-benzoyl-4-oxo-4H-chromen-6-yl)-N-(5-cyclopropyl-1H-pyrazol-3-yl)acetamide.

### Example 3

2-(4-methoxyphenyl)-6-methyl-4H-chromen-4-one

A solution of 3.15 g (0.021 mol) of 1-(2-hydroxy-5-methylphenyl)ethanone and 6.9 g (0.042 mol) of 1-methyl-4-methoxybenzoate in 50 ml of dry dioxane was added dropwise to a suspension of 50% NaH (2.96 g; 0.063 mol) in 40 ml of dry dioxane, under stirring at room temperature. After the addition, the reaction mixture was kept under reflux for 8 hours. After cooling, the solution was diluted with 150 ml of hexane and the precipitate was filtered off. The collected product was dissolved in 100 ml of water and then precipitated by acidification with acetic acid; after filtration and washing with water, 2.98 g (50% yield) of 1-(2-hydroxy-5-methylphenyl)-3-(4-methoxy-1,3-propandione were obtained and used for the next step without purification. A solution of 2.98 g (0.051 mol) of this intermediate in 70 ml of ethanol, containing concentrated HCl (2%) was refluxed for 2.5 hours. The solution was concentrated in vacuum to half volume and the precipitate was filtered off and washed with ethanol and then with water, giving 2.79 g (89% yield) of the title compound.

### Example 4

2-[2-(4-methoxyphenyl)-4-oxo-4H-chromen-6-yl]-propanoic acid

To a solution of 3 g (9.68 mmol) of [2-(4-methoxyphenyl)-4-oxo-4H-chromen-6-yl]acetic acid, prepared according to the method described in J. Indian. Chem. Soc. (1973), 295-298 [see also Eur. J. Med. Chem. (1978), 33-39], 20 ml of dry THF and 1.3 g (11.6 mmol) of ^{t}BuOK were added at −70° C. After 20 minutes under stirring, 0.72 ml (11.6 mmol) of MeI were added dropwise. The reaction mixture was maintained at the same temperature for 1.5 hours and then quenched with water. The resulting solution was acidified with HCl 2N and extracted with ethyl acetate. The organic layer was separated, washed with brine and dried over anhydrous Na₂SO₄ and concentrated in vacuum to give 2.19 g (70% yield) of the title compound.

### Example 5

N-(5-cyclopropyl-1H-pyrazol-3-yl)2-[2-(4-methoxyphenyl)4-oxo-4H-chromen-6-yl]propanamide;

2 g (6.17 mmol) of 2-[2-(4-methoxyphenyl)-4-oxo-4H-chromen-6-yl]-propanoic acid were suspended in 25 ml of CH₂Cl₂. 1.06 ml (6.17 mmol) of N,N-diisopropylethylamine and 1.16 (6.17 mmol) of N-ethyl-N′-diisopropylcarbodiimide were then added therein at 0° C. under stirring. After 20 minutes at this temperature, a solution of 691 mg (3.1 mmol) of tert-butyl-5-amino-3-cyclopropyl-1H-pyrazole-1-carboxylate, in 5 ml of CH₂Cl₂, was added dropwise. After 16 hours at room temperature, the mixture was washed with a saturated NaHCO₃ solution and then with water. The organic layer was dried over anhydrous Na₂SO₄ and evaporated to give 1.06 g (65% yield) of tert-butyl 3-cyclopropyl-5-({2-[2-(4-methoxyphenyl)4-oxo-4H-chromen-6-yl]propanoyl}amino)-1H-pyrazole-1-carboxylate. This intermediate, without further purification, was redissolved with 25 ml of a mixture 9/1 of CH₂Cl₂ and trifluoroacetic acid (TFA) and the resulting solution was maintained at room temperature for 2 hours. The solvent was then evaporated, CH₂Cl₂ added to the residue and the mixture washed with a saturated NaHCO₃ solution. The organic layer was dried over anhydrous Na₂SO₄ and concentrated under vacuum to give, after crystallisation from diisopropylether, 730 mg (85% yield) of the title compound.

### Example 6

2-(4-methoxyphenyl)-6-methyl-4(1H)-quinolinone

1 g (4.29 mmol) of 4-methyl-2-iodoaniline, 1.13 g (8.58 mmol) of 4-methoxy-phenylacetylene, 15 ml of diethylamine and 0.031 g (0.0429 mmol) of PdCl₂(dppf) were placed into a 100 ml stainless steel autoclave. The mixture was stirred and maintained under CO (20 atm) at 120° C. for 1 hour. After cooling, the formed product was collected by suction and purified by crystallization from ethanol yielding 0.79 g (70% yield) of the title compound.

### Example 7

2-(4-methoxyphenyl-6-methyl-4H-thiochromen-4-one

To a mixture of 1 g (8 mmol) of 4-methylbenzenthiol and 1.99 g (9.6 mmol) of methyl 3-(4-methoxyphenyl)-3-oxopropanoate, 15 ml of polyphosphoric acid were added. The solution was heated at 80° C. for 2 hours and then poured into icy water. The precipitated was filtered off and desiccated under vacuum yielding the title compound (1.62 g, 72% yield).

1) A method for treating cell proliferative disorders associated with an altered cell cycle dependent kinase activity, by administering to a mammal in need thereof an effective amount of a chromane derivative represented by formula 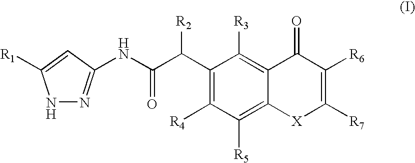
wherein
R₁ is a C₃-C₆ cycloalkyl group optionally substituted by a straight or branched C₁-C₆ alkyl or by aryl C₁-C₆ alkyl group;
R₂ is a hydrogen atom or a straight or branched C₁-C₆ alkyl or C₂-C₄ alkenyl group, each of which being optionally substituted by hydroxy, C₁-C₆ alkoxy, amino or C₁-C₆ alkylamino;
R₃, R₄ and R₅ are, each independently, hydrogen, halogen, hydroxy, amino or straight or branched C₁-C₆ alkyl, C₁-C₆ alkoxy or C₁-C₆ alkylamino;
P₆ and R₇ are, each independently, hydrogen, hydroxy, amino, aminocarbonyl, ureido, guanidyl, pyrrolidinyl optionally substituted by oxo groups, straight or branched C₁-C₆ alkyl optionally substituted by hydroxy or amino groups, straight or branched C₁-C₆ alkoxy, aryl or arylcarbonyl optionally substituted by halogen, hydroxy, amino, straight or branched C₁-C₆ alkyl or C₁-C₆ alkoxy groups, or a group selected from alkylcarbonyl, alkylamino, alkylaminocarbonyl or arylalkyloxy wherein alkyl stands for straight or branched C₁-C₆ alkyl;
X is an oxygen or sulfur atom or represents a group —N(R₈)— wherein R₈ is hydrogen
or a straight or branched C₁-C₆ allyl or C₂-C₄ alkenyl group, each of which being optionally substituted by hydroxy, amino, C₁-C₆ alkoxy or C₁-C₆ alkylamino;
or a pharmaceutically acceptable salt thereof;
provided that the compound of formula (I) is other than N-(5-cyclopropyl-1H-pyrazol-3-yl)-2-[2-(4-methoxyphenyl)-4-oxo-4H-chromen-6-yl]acetamide.

2) The method according to claim 1 wherein the cell proliferative disorder is selected from the group consisting of cancer, Alzheimer's disease, viral infections, auto-immune diseases and neurodegenerative disorders.

3) The method according to claim 2 wherein the cancer is selected from the group consisting of carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer, and Kaposi's sarcoma.

4) The method according to claim 1 wherein the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis, glomerulonephritis and post-surgical stenosis and restenosis.

5) The method according to claim 1 which provides tumor angiogenesis and metastasis inhibition.

6) The method according to claim 1 which provides treatment or prevention of radiotherapy-induced or chemotherapy-induced alopecia.

7) The method according to claim 1 further comprising subjecting the mammal in need thereof to a radiation therapy or chemotherapy regimen in combination with at least one cytostatic or cytotoxic agent.

8) The method according to claim 1 wherein the mammal in need thereof is a human.

9) A chromane derivative represented by formula 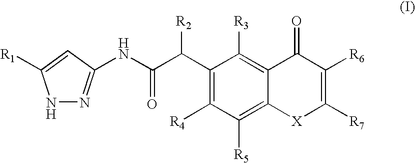
wherein
R₁ is a C₃-C₆ cycloalkyl group optionally substituted by a straight or branched C₁-C₆ alkyl or by aryl C₁-C₆ alkyl group;
R₂ is a hydrogen atom or a straight or branched C₁-C₆ alkyl or C₂-C₄ alkenyl group, each of which being optionally substituted by hydroxy, C₁-C₆ alkoxy, amino or C₁-C₆ alkylamino;
R₃, R₄ and R₅ are, each independently, hydrogen, halogen, hydroxy, amino or straight or branched C₁-C₆ alkyl, C₁-C₆ alkoxy or C₁-C₆ alkylamino;
R₆ and R₇ are, each independently, hydrogen, hydroxy, amino, aminocarbonyl, ureido, guanidyl, pyrrolidinyl optionally substituted by oxo groups, straight or branched C₁-C₆ alkyl optionally substituted by hydroxy or amino groups, straight or branched C₁-C₆ alkoxy, aryl or arylcarbonyl optionally substituted by halogen, hydroxy, amino, straight or branched C₁-C₆ alkyl or C₁-C₆ alkoxy groups, or a group selected from alkylcarbonyl, alkylamino, alkylaminocarbonyl or arylalkyloxy wherein alkyl stands for straight or branched C₁-C₆ alkyl;
X is an oxygen or sulfur atom or represents a group —N(R₈)— wherein R₈ is hydrogen or a straight or branched C₁-C₆ allyl or C₂-C₄ alkenyl group, each of which being optionally substituted by hydroxy, amino, C₁-C₆ alkoxy or C₁-C₆ alkylamino;
or a pharmaceutically acceptable salt thereof;
provided that the compound of formula (I) is other than N-(5-cyclopropyl-1H-pyrazol-3-yl)-2-[2-(4-methoxyphenyl)-4-oxo-4H-chromen-6-yl]acetamide.

10) A chromane derivative of formula (I), according to claim 9, wherein R₁ is a C₃-C₆ cycloalkyl group; R₂ is hydrogen or a straight or branched C₁-C₄ alkyl group; R₃, R₄ and R₅ are, each independently, hydrogen, halogen or a straight or branched C₁-C₆ alkyl or C₁-C₆ alkoxy group; R₆ and R₇ are, each independently, hydrogen, hydroxy, amino, aminocarbonyl, ureido, guanidyl, straight or branched C₁-C₄ alkyl optionally substituted by hydroxy or amino, straight or branched C₁-C₄ alkoxy, straight or branched C₁-C₄ alkylcarbonyl or alkylaminocarbonyl, arylcarbonyl, aryl C₁-C₄ alkyloxy, or aryl optionally substituted by halogen, amino, hydroxy or straight or branched C₁-C₄ alkyl or C₁-C₄ alkoxy; X is an oxygen or sulfur atom or represents a group —N(R₈)— wherein R₈ is hydrogen or a straight or branched C₁-C₆ alkyl or C₂-C₄ alkenyl group, each of which being optionally substituted by hydroxy, amino, C₁-C₆ alkoxy or C₁-C₆ alkylamino.

11) A chromane derivative of formula (I), according to claim 10, wherein R₁ is cyclopropyl; R₂ is hydrogen or methyl; R₃, R₄ and R₅ are, each independently, hydrogen, halogen, methyl or methoxy; R₆ and R₇ are, each independently, hydrogen or aryl groups optionally substituted by halogen, amino, hydroxy or straight or branched C₁-C₄ alkyl or C₁-C₄ alkoxy groups; X is an oxygen or sulfur atom or represents a group —N(R₈)— wherein R₈ is hydrogen or a straight or branched C₁-C₆ alkyl or C₂-C₄ alkenyl group, each of which being optionally substituted by hydroxy, amino, C₁-C₆ alkoxy or C₁-C₆ alkylamino.

12) A chromane derivative of formula (I), according to claim 11, wherein R₁ is cyclopropyl; R₂ is hydrogen or methyl; R₃, R₄ and R₅ are, each independently, hydrogen, fluorine, chlorine or bromine or a methoxy group; R₆ and R₇ are, each independently, hydrogen or aryl optionally further substituted as above indicated, wherein the aryl is selected from the group consisting of phenyl, pyrrolyl, pyrazoly, imidazolyl, furyl, thienyl or pyridyl; X is an oxygen or sulfur atom or represents a group —N(R₈)— wherein R₈ is hydrogen or a straight or branched C₁-C₆ alkyl or C₂-C₄ alkenyl group, each of which being optionally substituted by hydroxy, amino, C₁-C₆ alkoxy or C₁-C₆ alkylamino.

13) A chromane derivative of formula (I), according to claim 9, wherein X is an oxygen atom.

14) A chromane derivative of formula (I), according to claim 9, wherein X is a group —N(R)— wherein R₈ is hydrogen or a straight or branched C₁-C₆ alkyl or C₂-C₄ alkenyl group optionally substituted by hydroxy, amino, C₁-C₆ alkoxy or C₁-C₆ alkylamino.

15) A chromane derivative of formula (I), according to claim 14, wherein R₈ is hydrogen.

16) A chromane derivative of formula (I) according to claim 9, optionally in the form of a pharmaceutically acceptable salt, selected from the group consisting of:
1. N-(5-cyclobutyl-1H-pyrazol-3-yl)-2-[2-(4-methoxyphenyl)-4-oxo-4H-chromen-6-yl]acetamide;
2. N-(5-cyclopentyl-1H-pyrazol-3-yl)-2-[2-(4-methoxyphenyl)-4-oxo-4H-chromen-6-yl]acetamide;
3. N-(5-cyclopropyl-1H-pyrazol-3-yl)-2-[2-(4-methoxyphenyl)-4-oxo-1,4-dihydro-6-quinolinyl]acetamide;
4. N-(5-cyclobutyl-1H-pyrazol-3-yl)-2-[2-(4-methoxyphenyl)-4-oxo-1,4-dihydro-6-quinolinyl]acetamide;
5. N-(5-cyclopentyl-1H-pyrazol-3-yl)-2-[2-(4-methoxyphenyl)-4-oxo-1,4-dihydro-6-quinolinyl]acetamide;
6. N-(5-cyclopropyl-1H-pyrazol-3-yl)-2-[2-(4-methoxyphenyl)-4-oxo-4H-chromen-6-yl]propanamide;
7. N-(5-cyclobutyl-1H-pyrazol-3-yl)-2-[2-(4-methoxyphenyl)-4-oxo-4H-chromen-6-yl]propanamide;
8. N-(5-cyclopentyl-1H-pyrazol-3-yl)-2-[2-(4-methoxyphenyl)-4-oxo-4H-chromen-6-yl]propanamide;
9. N-(5-cyclopropyl-1H-pyrazol-3-yl)-2-[2-(4-methoxyphenyl)-4-oxo-1,4-dihydro-6-quinolinyl]propanamide;
10. N-(5-cyclobutyl-1H-pyrazol-3-yl)-2-[2-(4-methoxyphenyl)-4-oxo-1,4-dihydro-6-quinolinyl]propanamide;
11. N-(5-cyclopentyl-1H-pyrazol-3-yl)-2-[2-(4-methoxyphenyl)-4-oxo-1,4 dihydro-6-quinolinyl]propanamide;
12. N-(5-cyclopropyl-1H-pyrazol-3-yl)-2-[2-(4-methoxyphenyl)-4-oxo-4H-thiochromen-6-yl]acetamide;
13. N-(5-cyclobutyl-1H-pyrazol-3-yl)-2-[2-(4-methoxyphenyl)-4-oxo-4H-thiochromen-6-yl]acetamide;
14. N-(5-cyclopentyl-1H-pyrazol-3-yl)-2-[2-(4-methoxyphenyl)-4-oxo-4H-thiochromen-6-yl]acetamide;
15. N-(5-cyclopropyl-1H-pyrazol-3-yl)-2-[8-fluoro-2-(4-methoxyphenyl)-4-oxo-4H-chromen-6-yl]acetamide;
16. N-(5-cyclopropyl-1H-pyrazol-3-yl)-2-[5-fluoro-2-(4-methoxyphenyl)-4-oxo-4H-chromen-6-yl]acetamide;
17. N-(5-cyclopropyl-1H-pyrazol-3-yl)-2-[7-fluoro-2-(4-methoxyphenyl)-4-oxo-4H-chromen-6-yl]acetamide;
18. 2-[8-chloro-2-(4-methoxyphenyl)-4-oxo-4H-chromen-6-yl]-N-(5-cyclopropyl-1H-pyrazol-3-yl)acetamide;
19. 2-[5-chloro-2-(4-methoxyphenyl)-4-oxo-4H-chromen-6-yl]-N-(5-cyclopropyl-1H-pyrazol-3-yl)acetamide;
20. 2-[7-chloro-2-(4-methoxyphenyl)-4-oxo-4H-chromen-6-yl]-N-(5-cyclopropyl-1H-pyrazol-3-yl)acetamide;
21. 2-[8-bromo-2-(4-methoxyphenyl)-4-oxo-4H-chromen-6-yl]-N-(5-cyclopropyl-1H-pyrazol-3-yl)acetamide;
22. 2-[5-bromo-2-(4-methoxyphenyl)-4-oxo-4H-chromen-6-yl]-N-(5-cyclopropyl-1H-pyrazol-3-yl)acetamide;
23. 2-[7-bromo-2-(4-methoxyphenyl)-4-oxo-4H-chromen-6-yl]-N-(5-cyclopropyl-1H-pyrazol-3-yl)acetamide;
24. N-(5-cyclopropyl-1H-pyrazol-3-yl)-2-[5-methoxy-2-(4-methoxyphenyl)-4-oxo-4H-chromen-6-yl]acetamide;
25. N-(5-cyclopropyl-1H-pyrazol-3-yl)-2-[8-methoxy-2-(4-methoxyphenyl)-4-oxo-4H-chromen-6-yl]acetamide;
26. N-(5-cyclopropyl-1H-pyrazol-3-yl)-2-[7-methoxy-2-(4-methoxyphenyl)-4-oxo-4H-chromen-6-yl]acetamide;
27. N-(5-cyclopropyl-1H-pyrazol-3-yl)-2-[2-(3-methoxyphenyl)-4-oxo-4H-chromen-6-yl]acetamide;
28. N-(5-cyclopropyl-1H-pyrazol-3-yl)-2-[2-(3-hydroxyphenyl)-4-oxo-4H-chromen-6-yl]acetamide;
29. 2-[2-(3-aminophenyl)-4-oxo-4H-chromen-6-yl]-N-(5-cyclopropyl-1H-pyrazol-3-yl)acetamide;
30. N-(5-cyclopropyl-1H-pyrazol-3-yl)-2-[2-(4-hydroxyphenyl)-4-oxo-4H-chromen-6-yl]acetamide;
31. 2-[2-(4-aminophenyl)-4-oxo-4H-chromen-6-yl]-N-(5-cyclopropyl-1H-pyrazol-3-yl)acetamide;
32. 2-[2-(4-chlorophenyl)-4-oxo-4H-chromen-6-yl]-N-(5-cyclopropyl-1H-pyrazol-3-yl)acetamide;
33. 2-[2-(4-bromophenyl)-4-oxo-4H-chromen-6-yl]-N-(5-cyclopropyl-1H-pyrazol-3-yl)acetamide;
34. N-(5-cyclopropyl-1H-pyrazol-3-yl)-2-[2-(4-fluorophenyl)-4-oxo-4H-chromen-6-yl]acetamide;
35. 2-[2-(3-chlorophenyl)-4-oxo-4H-chromen-6-yl]-N-(5-cyclopropyl-1H-pyrazol-3-yl)acetamide;
36. 2-[2-(3-bromophenyl)-4-oxo-4H-chromen-6-yl]-N-(5-cyclopropyl-1H-pyrazol-3-yl)acetamide;
37. N-(5-cyclopropyl-1H-pyrazol-3-yl)-2-[2-(3-fluorophenyl)-4-oxo-4H-chromen-6-yl]acetamide;
38. 6-{2-[(5-cyclopropyl-1H-pyrazol-3-yl)amino]-2-oxoethyl}-4-oxo-4H-chromene-2-carboxamide;
39. 6-{2-[(5-cyclopropyl-1H-pyrazol-3-yl)amino]-2-oxoethyl}-N-methyl-4-oxo-4H-chromene-2-carboxamide;
40. N-(5-cyclopropyl-1H-pyrazol-3-yl)-2-[4-oxo-2-(1H-pyrrol-2-yl)-4H-chromen-6-yl]acetamide;
41. N-(5-cyclopropyl-1H-pyrazol-3-yl)-2-[4-oxo-2-(1H-pyrazol-5-yl)-4H-chromen-6-yl]acetamide;
42. N-(5-cyclopropyl-1H-pyrazol-3-yl)-2-[2-(1H-imidazol-5-yl)-oxo-4H-chromen-6-yl]acetamide;
43. N-(5-cyclopropyl-1H-pyrazol-3-yl)-2-[4-oxo-2-(5-oxo-2-pyrrolidinyl)-4H-chromen-6-yl]acetamide;
44. N-(5-cyclopropyl-1H-pyrazol-3-yl)-2-[2-(2-furyl)-4-oxo-4H-chromen-6-yl]acetamide;
45. N-(5-cyclopropyl-1H-pyrazol-3-yl)-2-[2-(3-furyl)-4-oxo-4H-chromen-6-yl]acetamide;
46. N-(5-cyclopropyl-1H-pyrazol-3-yl)-2-[4-oxo-2-(3-thienyl)-4H-chromen-6-yl]acetamide;
47. N-(5-cyclopropyl-1H-pyrazol-3-yl)-2-[4-oxo-2-(2-thienyl)-4H-chromen-6-yl]acetamide;
48. N-(5-cyclopropyl-1H-pyrazol-3-yl)-2-[4-oxo-2-(2-pyridinyl)-4H-chromen-6-yl]acetamide;
49. N-(5-cyclopropyl-1H-pyrazol-3-yl)-2-[4-oxo-2-(3-pyridinyl)-4H-chromen-6-yl]acetamide;
50. N-(5-cyclopropyl-1H-pyrazol-3-yl)-2-(2-hydroxy-4-oxo-4H-chromen-6-yl)acetamide;
51. 2-(2-amino-4-oxo-4H-chromen-6-yl)-N-(5-cyclopropyl-1H-pyrazol-3-yl)acetamide;
52. 2-{2-[(aminocarbonyl)amino]-4-oxo-4H-chromen-6-yl}-N-(5-cyclopropyl-1H-pyrazol-3-yl)acetamide;
53. 2-(2-{[amino(imino)methyl]amino}-4-oxo-4H-chromen-6-yl)-N-(5-cyclopropyl-1H-pyrazol-3-yl)acetamide;
54. N-(5-cyclopropyl-1H-pyrazol-3-yl)-2-(2-hydroxy-4-oxo-1,4-dihydro-6-quinolinyl)acetamide;
55. 6-{2-[(5-cyclopropyl-1H-pyrazol-3-yl)amino]-2-oxoethyl}-4-oxo-1,4-dihydro-2-quinolinecarboxamide;
56. 6-{2-[(5-cyclopropyl-1H-pyrazol-3-yl)amino]-2-oxoethyl}-N-methyl-4-oxo-1,4-dihydro-2-quinolinecarboxamide;
57. N-(5-cyclopropyl-1H-pyrazol-3-yl)-2-[4-oxo-2-(1H-pyrrol-2-yl)-1,4-dihydro-6-quinolinyl]acetamide;
58. N-(5-cyclopropyl-1H-pyrazol-3-yl)-2-[4-oxo-2-(1H-pyrazol-5-yl)-1,4-dihydro-6-quinolinyl]acetamide;
59. N-(5-cyclopropyl-9H-pyrazol-3-yl)-2-[2-(1H-imidazol-5-yl)-4-oxo-1,4-dihydro-6-quinolinyl]acetamide;
60. N-(5-cyclopropyl-1H-pyrazol-3-yl)-2-[2-(4-methoxyphenyl)-1-methyl-4-oxo-1,4-dihydro-6-quinolinyl]acetamide;
61. N-(5-cyclopropyl-1H-pyrazol-3-yl)-2-[1-(2-hydroxyethyl)-2-(4-methoxyphenyl)-4-oxo-1,4-dihydro-6-quinolinyl]acetamide;
62. 2-[1-(2-aminoethyl)-2-(4-methoxyphenyl)-4-oxo-1,4-dihydro-6-quinolinyl]-N-(5-cyclopropyl-1H-pyrazol-3-yl)acetamide;
63. N-(5-cyclopropyl-1H-pyrazol-3-yl)-2-[2-(4-methoxyphenyl)-3-methyl-4-oxo-4H-chromen-6-yl]acetamide;
64. N-(5-cyclopropyl-1H-pyrazol-3-yl)-2-(4-oxo-4H-chromen-6-yl)acetamide;
65. N-(5-cyclopropyl-1H-pyrazol-3-yl)-2-(4-oxo-1,4-dihydro-6-quinolinyl)acetamide;
66. N-(5-cyclopropyl-1H-pyrazol-3-yl)-2-(4-oxo-4H-thiochromen-6-yl)acetamide;
67. N-(5-cyclopropyl-1H-pyrazol-3-yl)-2-[3-hydroxy-2-(4-methoxyphenyl)-4-oxo-4H-chromen-6-yl]acetamide;
68. N-(5-cyclopropyl-1H-pyrazol-3-yl)-2-[3-methoxy-2-(4-methoxyphenyl)-4-oxo-4H-chromen-6-yl]acetamide;
69. 2-[3-(benzyloxy)-2-(4-methoxyphenyl)-4-oxo-4H-chromen-6-yl]-N-(5-cyclopropyl-1H-pyrazol-3-yl)acetamide;
70. 6-{2-[(5-cyclopropyl-1H-pyrazol-3-yl)amino]-2-oxoethyl}-2-(4-methoxyphenyl)-4-oxo-4H-chromene-3-carboxamide;
71. 2-(3-acetyl-4-oxo-4H-chromen-6-yl)-N-(5-cyclopropyl-1H-pyrazol-3-yl)acetamide;
72. 2-(3-benzoyl-4-oxo-4H-chromen-6-yl)-N-(5-cyclopropyl-1H-pyrazol-3-yl)acetamide.

17) A process for preparing the compounds of formula (I) or the pharmaceutically acceptable salts thereof, as defined in claim 9, which process comprises:
a) racting the compounds of formula (II) or the regioisomers of formula (IIa) 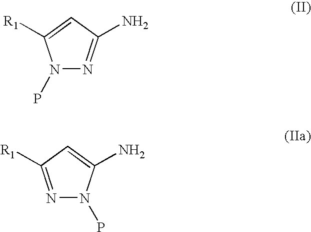
wherein R₁ is as defined in claim 9 and P represents a suitable nitrogen-pyrazole protecting group, with the compounds of formula 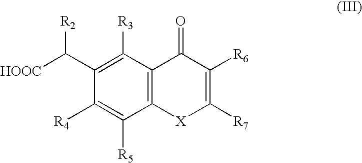
wherein R₂, R₃, R₄, R₅, R₆, R₇ and X are as defined in claim 9, thus obtaining the compounds of formula (I) or (IVa) 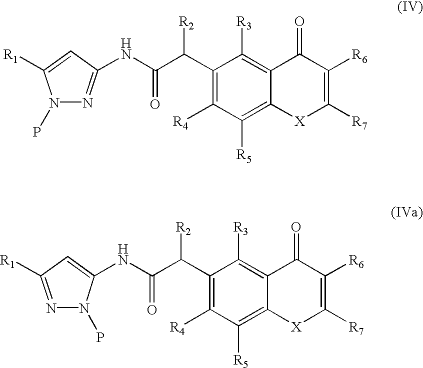
b) and deprotecting the compounds of formula (IV) or (IVa) so as to obtain the derivatives of formula (I) and, if desired, converting them into pharmaceutically acceptable salts thereof.

18) The process of claim 17 wherein, within the compounds of formula (II) or (IIa), P represents the group tert-butoxycarbonyl.

19) A pharmaceutical composition comprising a therapeutically effective amount of a chromane derivative of formula (I), as defined in claim 9, and at least one pharmaceutically acceptable excipient, carrier and/or diluent.

20) A pharmaceutical composition according to claim 19 further comprising one or more chemotherapeutic agents, as a combined preparation for simultaneous, separate or sequential use in anticancer therapy.

21) A product or kit comprising a compound of formula (I) as defined in claim 9 or a pharmaceutical composition thereof as defined in claim 19, and one or more chemotherapeutic agents, as a combined preparation for simultaneous, separate or sequential use in anticancer therapy.

22) A compound of formula (I), as defined in claim 9, for us as a medicament.

23) Use of a compound of formula (I), as defined in claim 9, in the manufacture of a medicament for the treatment of cell proliferative disorders associated with an altered cell cycle dependent kinase activity.

24) Use according to claim 23 for treating tumors.
